(19) United States
(12) Patent Application Publication
(10) Pub. No.: US 2009/0275804 A1
(43) Pub. Date: Nov. 5, 2009

(54) Hinged Retractor With Sheath

(76) Inventors: Rudolf Bertagnoli, Straubing (DE); Steve Nowak, Traverse City, MI (US); Christopher T. Martin, Empire, MI (US); Daniel K. Farley, Traverse City, MI (US); Josh Delickta, Elk Rapids, MI (US)

Correspondence Address: MCANDREWS HELD & MALLOY, LTD, 500 WEST MADISON STREET, SUITE 3400, CHICAGO, IL, 60661, US

(21) Appl. No.: 12/433,226

(22) Filed: Apr. 30, 2009

Related U.S. Application Data
(60) Provisional application No. 61/049,205, filed on Apr. 30, 2008.
(60) Provisional application No. 61/105,137, filed on Oct. 14, 2008.

(51) Int. Cl. A61B 1/32

(52) U.S. Cl. 600/226; 600/201

(57) ABSTRACT

A hinged retractor includes a distal portion adapted to be located inside the body of a patient during a surgical procedure and a proximal portion adapted to be closer to the surgeon during a surgical procedure. The hinged retractor includes a first side including a first blade and a second side including a second blade. The second side is joined to the first side proximate to a distal end of the hinged retractor. The hinged retractor includes a pivot proximate to the distal end. The hinged retractor is movable between a closed position and an open position, wherein a distance between a proximal portion of the first and second blades increases more than a distance between a distal portion of the first and second blades when the hinged retractor is moved from the closed position to the open position.

### CROSS-REFERENCE TO RELATED APPLICATION

The present application makes reference to, claims priority to, and claims benefit of U.S. Provisional Application No. 61/049205, entitled “Hinged Retractor with Sheath,” Attorney Docket No. 19670US02, filed Apr. 30, 2008, the complete subject matter of which is hereby incorporated herein by reference, in its entirety. The present application also makes reference to, claims priority to, and claims benefit of U.S. Provisional Application No. 61/105137, entitled “Retractor with Sheath,” Attorney Docket No. 20005US01, filed Oct. 14, 2008, the complete subject matter of which is hereby incorporated herein by reference, in its entirety.

### STATEMENT REGARDING FEDERALLY SPONSORED RESEARCH OR DEVELOPMENT

None.

### BACKGROUND OF THE INVENTION

The present invention relates to surgical apparatus for retracting anatomy to provide exposure of an operative site, and more particularly relates to a retraction apparatus having a hinged or pivoting distal end.

In surgical operations, retraction devices are used to properly access internal organs and bone structures. Retraction devices are generally designed to hold back the anatomy in the immediate area of the operative site to enable a surgeon to have both an optimal view of the site and a sufficiently-open area within which to work. During a surgical procedure, a surgeon will typically make an incision in a patient to access the sites of interest, such as an internal organ or organs, and/or bone structures, depending on the procedure. A retraction device may then be used to maintain clear access to the site of interest.

For example, during a spinal fixation procedure, screws are inserted into a patient's vertebrae, typically at the pedicles. A rod may then be secured to the screws, thereby helping to fix a particular potion of the spinal column in place. To perform such a procedure, access to the spinal column is needed. A retractor may be used to hold back tissue and allow a surgeon access to place one or more screws and/or connecting rods, and/or perform other procedures on the spinal column. Ideally, such access would include room to manipulate the various surgical implements required, as well as good lighting and visibility. Further, it may be desirable for such access to allow for adjustability to accommodate different patients or procedures. Moreover, it may desirable to have the ability to maneuver around a placed rod and/or other obstruction at the access site.

It is therefore one object of the present invention to provide a surgical retractor that provides improved access, and/or visibility, and/or adjustability, and/or maneuverability around a surgical site of interest.

### BRIEF SUMMARY OF THE INVENTION

These and other objects of the invention are achieved in a hinged retractor. In one embodiment of the present invention a hinged retractor having a distal portion adapted to be located inside the body of a patient during a surgical procedure and a proximal portion adapted to be closer to the surgeon during a surgical procedure is provided. The hinged retractor includes a first side including a first blade and a second side including a second blade. The second side is joined to the first side proximate to a distal end of the hinged retractor. The hinged retractor includes a pivot proximate to the distal end. The hinged retractor is movable between a closed position and an open position, wherein a distance between a proximal portion of the first and second blades increases more than a distance between a distal portion of the first and second blades when the hinged retractor is moved from the closed position to the open position.

In certain embodiments, the hinged retractor includes a sheath that is attachable to and removable from the first and second blades. The sheath surrounds at least a portion of the distal portion of the hinged retractor when attached to the first and second blades. The sheath is expandable with the hinged retractor when the hinged retractor is moved from the closed position to the open position and collapsible with the hinged retractor when the hinged retractor is moved from the open position to the closed position.

In certain embodiments, the hinged retractor includes a plurality of depth indicators. The depth indicators indicate positions at which at least one of the first and second blades may be bent to adapt the hinged retractor for a procedure. Further, in certain embodiments, at least one of the first and second blades includes bendable teeth located proximate to the distal end of the hinged retractor. At least one of the bendable teeth may include an opening adapted for the insertion of a bending tool for manipulating the at least one of the bendable teeth. In certain embodiments, the first and second blade may be removably joined.

In one embodiment of the present invention a retractor system is provided. The retractor system includes a hinged retractor and a blade spreader. The hinged retractor has a distal portion adapted to be located inside the body of a patient during a surgical procedure and a proximal portion adapted to be closer to the surgeon during a surgical procedure. The hinged retractor includes a first side including a first blade and a second side including a second blade. The second side is joined to the first side proximate to a distal end of the hinged retractor. The hinged retractor includes a pivot proximate to the distal end. The hinged retractor is movable between a closed position and an open position, wherein a distance between a proximal portion of the first and second blades increases more than a distance between a distal portion of the first and second blades when the hinged retractor is moved from the closed position to the open position. The blade spreader is securable to the first and second blades, and is adapted to maintain the hinged retractor in the open position during a procedure. The blade spreader is securable to a frame to secure the retractor system in place. The blade spreader may further include a first adjustment portion adapted to open the hinged retractor and a second adjustment portion adapted to position the hinged retractor relative to the frame.

The retractor system may further include a sheath attachable to and removable from the first and second blades. In certain embodiments, the retractor system includes a sheath spreader adapted to expand the sheath to facilitate attachment of the sheath to the first and second blades. Further, the first and second blades may include bendable teeth proximate to the distal end of the hinged retractor. In certain embodiments, the retractor system includes a teeth bending tool. The bendable teeth may include an opening adapted for insertion of the teeth bending tool for bending the bendable teeth.

In certain embodiments, the retractor system includes a gauge that includes a plurality of gauge depth indicators that are adapted to determine a bending location of at least one of the first and second blades. At least one of the first and second blades includes a plurality of blade depth indicators corresponding to the gauge depth indicators. The plurality of blade depth indicators indicate positions at which the at least one of the first and second blades may be bent to adapt the hinged retractor for a procedure. The retractor system may further include a blade bender adapted to facilitate bending of at least one of the first and second blades.

In one embodiment of the present invention, a method of providing access to a surgical incision with a hinged retractor is provided. The method includes positioning the hinged retractor, in a closed position, inside the patient. The method also includes adjusting the hinged retractor to an open position and securing the hinged retractor in place. In certain embodiments, the method may include surrounding at least a portion of the distal portion of the hinged retractor with an expandable sheath before positioning the hinged retractor inside the patient. Further, the method may comprise bending at least one bendable tooth proximate to the distal end of the hinged retractor after the hinged retractor is positioned inside the patient. The method may also comprise using a gauge to determine an appropriate hinged retractor before positioning the hinged retractor inside the patient.

### BRIEF DESCRIPTION OF THE SEVERAL VIEWS OF THE DRAWINGS

FIG. 1 provides a top view of a hinged retractor system formed in accordance with an embodiment of the present invention.

FIG. 2 illustrates a perspective view of a hinged retractor formed in accordance with an embodiment of the present invention.

FIG. 3 provides a perspective view of a blade for a hinged retractor formed in accordance with an embodiment of the present invention.

FIG. 4 provides a perspective view of the blade of FIG. 3 after the blade has been bent to suit a procedure.

FIG. 5 illustrates a top view of a blade spreader formed in accordance with the present invention.

FIG. 6 illustrates a perspective view of a sheath formed in accordance with an embodiment of the present invention.

FIG. 7 illustrates a side view of a sheath in place on a hinged retractor in accordance with an embodiment of the present invention.

FIG. 8 illustrates a side view of a sheath spreader formed in accordance with an embodiment of the present invention.

FIG. 9 illustrates a blade gauge set formed in accordance with an embodiment of the present invention.

FIG. 10 illustrates a blade bender formed in accordance with an embodiment of the present invention.

FIG. 11 illustrates a teeth bender set formed in accordance with an embodiment of the present invention.

FIG. 12 illustrates a blade manipulator formed in accordance with an embodiment of the present invention.

### DETAILED DESCRIPTION

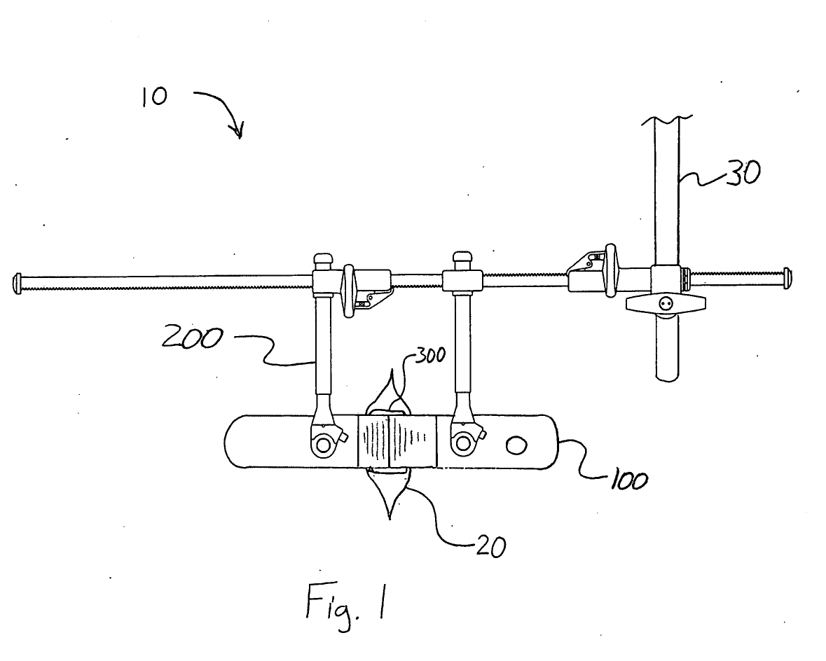

FIG. 1 presents a top view of a hinged retractor system 10 formed in accordance with an embodiment of the present invention. The hinged retractor system 10 is used during a surgical procedure to provide access to an incision 20. For example, the hinged retractor system 10 may provide access to a patient's spine during a spinal fixation procedure. The hinged retractor system 10 illustrated in FIG. 1 includes a frame 30, a hinged retractor 100, a blade spreader 200, and a sheath 300.

The frame 30 is securely mounted to an external mount, such as an operating table (not shown), to secure the hinged retractor system 10 in place during a surgical procedure. In the illustrated embodiment, the frame 30 is secured to the blade spreader 200 to maintain the hinged retractor 100 in place. The blade spreader 200 acts to open the hinged retractor 100 and secure the hinged retractor 100 in a desired position to provide access to a surgical site of interest via the incision 20.

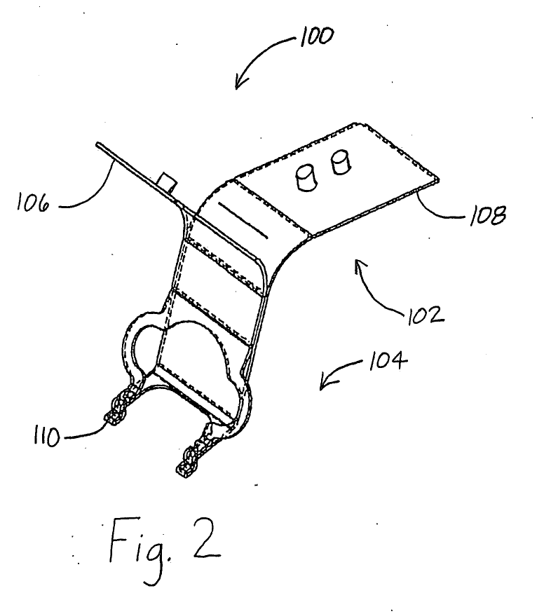

FIG. 2 illustrates a perspective view of a hinged retractor 100 formed in accordance with an embodiment of the present invention. The hinged retractor 100 includes a proximal portion 102 and a distal portion 104. The distal portion 104 is the portion of the hinged retractor 100 oriented most deeply inside the body of a patient when the hinged retractor 100 is in place during a surgical procedure, and the proximal portion 102 is the portion of the hinged retractor 100 located closer to the surgeon performing the procedure and/or outside of the body of the patient.

The hinged retractor 100 includes a first blade 106 and a second blade 108 joined by a pivot 110. In the illustrated embodiment, the hinged retractor 100 is formed from a single piece, and the pivot 110 is formed by bending. Alternatively, a hinged retractor could be formed from two or more pieces, and/or the first and second blades could be removably joined proximate to the pivot 110. As one example, the first blade 106 could include a tab accepted by either a hole or a slot in the second blade 108. In FIG. 2, the hinged retractor 100 is shown in a closed position, with the first blade 106 and second blade 108 at or near in contact with one another. To move the hinged retractor 100 to an open position, the first blade 106 and second blade 108 are drawn away from each other, causing the blades to bend about pivot 110, resulting in the blades being farther apart at the proximal portion 102 of the hinged retractor 100 than at the distal portion 104.

The first blade 106 in the embodiment illustrated in FIG. 2 is adapted to be placed proximate to the spine in a spinal fixation procedure. The first blade 106 (as well as the second blade 108) provides a surface to hold back tissue from the access site during surgery, by contacting the tissue and/or supporting a sheath, discussed further below. The first blade 106 may be configured to be fit around the Facet Capsule of a patient and reach at or near the lamina to help provide access to a pedicle for implanting a screw, for example. In the illustrated embodiment, the first blade 106 includes semicircles around its perimeter that are adapted to fit around the Facet Capsule when the hinged retractor 100 is used during a surgical procedure.

The hinged retractor 100 of the embodiment illustrated in FIG. 2 may be formed, for example, from a single piece of grade 1 titanium with a thickness of around 0.063 inches. A piece of titanium corresponding to the overall length and width of the hinged retractor may first be provided. Then, the shapes of the cutouts and the pivot 110 may be formed by cutting away material. Finally, the remaining material may be put in a die and bent to shape. The portions of the first blade 106 and second blade 108 at the tip of the distal portion 104 may be crimped, welded, or hemmed together as part of the forming process. The portions of the first blade 106 and second blade 108 at the tip of the distal portion 104 may further be formed to be adapted to engage the anterior surface of the lamina. The first blade 106 and second blade 108 are shaped to provide a sufficient amount of access to the site of interest when the hinged retractor 100 is in an open position, while still maintaining a sufficiently narrow profile for ease of insertion into a patient when the hinged retractor 10 is in the closed position. The size and shape of the first blade 106 and second blade 108 are designed to accommodate the goals of providing sufficient access in the open position, providing sufficient ease of insertion in the closed position, and providing adaptability and ease of manipulation for use in different patients and/or procedures. The material used to form the hinged retractor 100, as well as widths and thicknesses, should allow for sufficient malleability, in that the hinged retractor 100 formed as described above will be bent to shape in the manufacturing process and/or may further be bent or re-shaped during a procedure, but should also provide for sufficient rigidity that the hinged retractor 10, including the handles and blades, maintains its shape when in use during surgery.

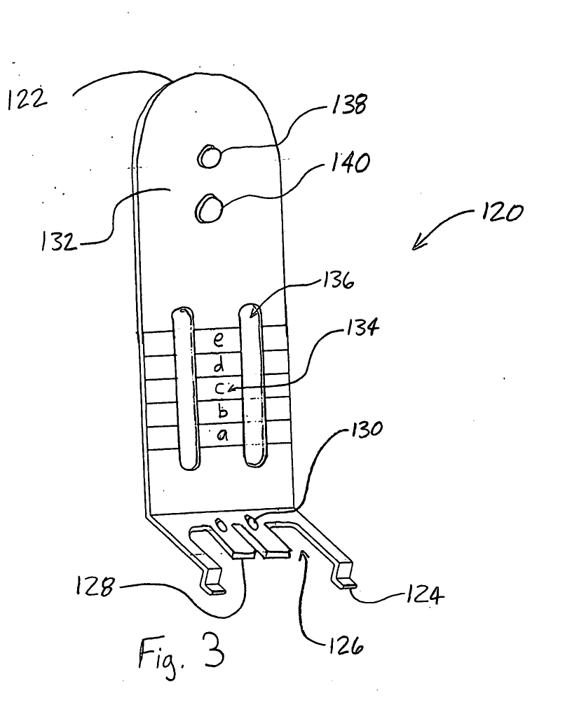

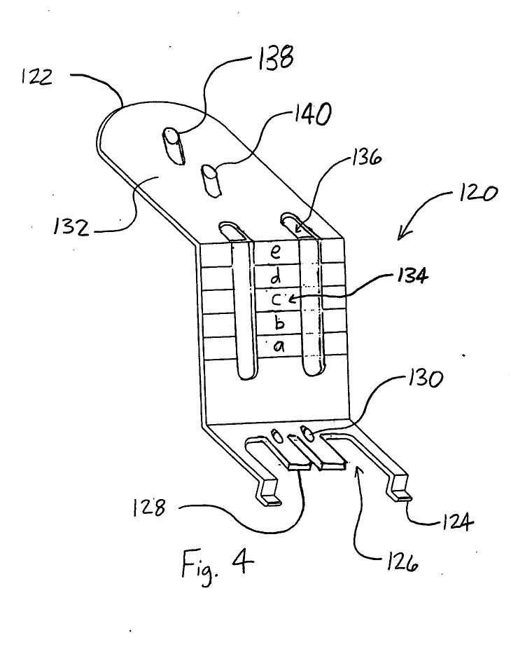

FIG. 3 provides a perspective view of a blade 120 formed in accordance with an embodiment of the present invention, and FIG. 4 provides a perspective view of the blade 120 of FIG. 3 after the blade 120 has been bent to suit a procedure. The blade 120 illustrated in FIGS. 3-4 corresponds to a first blade as described above, but the following description of a blade also generally applies to a second blade in many respects. As would be appreciated by one skilled in the art, the shapes of the cutouts in the first and second blades would generally be different to accomplish the goals described above.

The blade 120 illustrated in FIGS. 3-4 includes a proximal end 122 and a distal end 124 (the above definition regarding “proximal” and “distal” also applies here). The blade 120 also includes a cutout 126, teeth 128, bending tool openings 130, a handle 132, depth indicators 134, bending cutouts 136, a handling member 138, and a securement member 140.

The cutout 126 is sized and adapted to provide access to the surgical site of interest. The cutout 126 is located proximate to the distal end 124, and, as illustrated in the embodiment of FIGS. 3-4, may extend to the distal end 124, to provide access, for example, to the pedicles of a patient's spine. For example, the cutout 126 may be sized and adapted to allow access to two screws for attaching a rod in a cervical fixation procedure. As an example of one alternative, the cutout may be sized and adapted to allow access to a single screw. As an example of a further alternative, the cutout may be open on one end to facilitate removal of a hinged retractor after a rod has been installed. Further, as the access desired at the site of interest may be different at the location of a first blade or a second blade, the cutouts may be different for the first and second blades of a hinged retractor.

The teeth 128 are located proximate to the distal end 124 and the cutout 126. The teeth 128 are of a suitable thickness and shape to permit a surgeon to bend the teeth 128 during a procedure. The teeth 128 are illustrated in FIGS. 3-4 in an original, or unbent, orientation. By bending the teeth 128 after insertion, the access to the site may be adjusted and increased, while allowing easier insertion and placement of a hinged retractor with the teeth 128 in an original, or un-bent orientation. In the illustrated embodiment, the blade 120 includes two teeth 128. Alternatively, a different number of teeth could be employed. Further, the first and second blades of a hinged retractor may have different numbers of teeth. For example, the first blade of a hinged retractor may have two teeth and the second blade may have five teeth. Bending tool openings 130 are located proximate to the teeth 128. In the illustrated embodiment, each tooth 128 has a corresponding bending tool opening 130. The bending tool openings 130 are sized and adapted to accept a bending tool to facilitate bending of the teeth 128.

The handle 132 is located proximate to the proximal end 122 of the blade 120. The handle 132 is sized and adapted to provide a convenient location for grasping, positioning, and/or manipulating the blade 120.

The depth indicators 134 are indicated by the letters a-e. In certain embodiments of the present invention, the depth indicators 134 mark distances of 50, 60, 70, 80, and 90 millimeters, respectively, from the tip of the distal end 124 of the blade 120. The depth indicators 134 provide a visual indication of locations along the blade 120 to allow pre-bending of the blade 120 for a particular procedure. For example, a gauge may be used (discussed below) to identify a desired portion of the blade 120 to be bent before insertion into a patient for a particular procedure. The blade 120 may be pre-bent so that the handle 132 is aligned horizontally slightly above the top of the surface of the patient's body when the blade 120 is in place for the procedure. In the illustrated embodiment, the blade 120 includes bending cutouts 136 proximate to the depth indicators 134. The bending cutouts 136 are sized and adapted to remove material from the blade 120 proximate to the portions that may be bent to facilitate easier bending.

The handling member 138 provides a location for grasping the blade 120 with a manipulating tool (discussed below). In the illustrated embodiment, the handling member 138 is a post extending from a surface of the handle 132. Use of a manipulating tool to grasp the handling member 138 may improve the ability of a surgeon to place the blade 120 compared to only handling the handle 132. In certain embodiments of the present invention, one of the first and second blades will include a handling member 138, and the other of the first and second blades will not.

The securement member 140 provides a location for securing the blade 120 to a frame or other assembly, such as a blade spreader (discussed below) for opening a hinged retractor and/or holding the blade 120 in a desired position during a procedure. In the illustrated embodiment, the securement member 140 is a post extending from a surface of the handle 132.

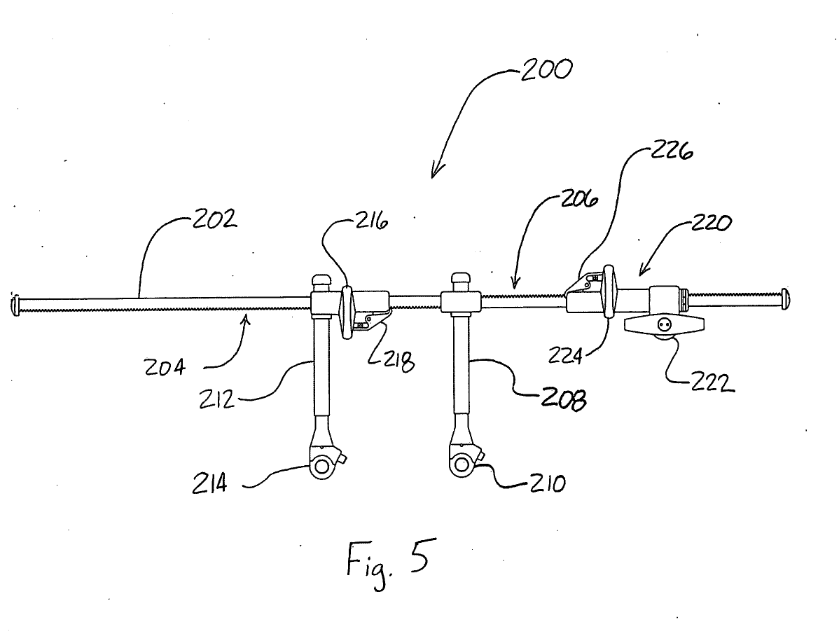

FIG. 5 illustrates a top view of a blade spreader 200 formed in accordance with the present invention. The blade spreader includes a spreader bar 202, a first set of teeth 204, and second set of teeth 206, a fixed arm 208, and adjustable arm 212, and a securement assembly 220. The blade spreader 200 of the illustrated embodiment is used to open a hinged retractor and maintain the hinged retractor in a desired position during a procedure. As shown in FIG. 5, in the illustrated embodiment, the first set of teeth 204 extend from one side of the spreader bar 202, and the second set of teeth 206 extend from an opposite side of the spreader bar 202. The spreader bar 202 of the illustrated embodiment is sized to span the torso of a patient during a medical procedure.

In the illustrated embodiment, the fixed arm 208 is interposed between the first set of teeth 204 and the second set of teeth 206. The fixed arm 208 is fixed in place in a position approximately midway along the length of the spreader bar 202. Toward the free end of the fixed arm 208, the blade spreader includes a fixed arm clamp 210. The fixed arm clamp 210 is sized and oriented to accept and secure a portion of a hinged retractor, such as the securement member 140 of the blade 120.

The blade spreader 200 also includes an adjustable arm 212. The adjustable arm 212 includes an adjustable arm clamp 214 that is generally similar to the fixed arm clamp 210. The adjustable arm 212 includes an adjustable arm thumbwheel 216 and adjustable arm lock 218. The adjustable arm thumbwheel 216 is used to adjust the position of the adjustable arm 212 along the length of the spreader bar 202. For example, in the illustrated embodiment, the adjustable arm thumbwheel 216 actuates a gear (not shown) that cooperates with the first set of teeth 204 to form a rack and pinion. When the adjustable arm thumbwheel 216 is turned, the adjustable arm 212 moves along the first set of teeth 204. Thus, the blade spreader 200 includes an opening mechanism to open the blades of a hinged retractor. In alternate embodiments, other devices may be employed to provide an opening mechanism, and move and/or position an adjustable arm along the length of a spreader bar. For example, the adjustable arm may be manually slid along the length, and secured in place with a clamp, or a pin. The adjustable arm 212 also includes an adjustable arm lock 218. The adjustable arm lock 218 serves to secure the adjustable arm 212 in place along the spreader bar 202. The adjustable arm lock 218 may function as a ratcheting mechanism. For example, in the illustrated embodiment, the adjustable arm lock 218 may rotate about a pivot, and be urged by a spring into place against the first set of teeth 204. The shape of the portion of the adjustable arm lock 218 may be such that it allows the adjustable arm lock to pivot against the spring when the adjustable arm 212 is urged in one direction, but not pivot against the spring when the adjustable arm 212 is urged in the opposite direction. For example, in the illustrated embodiment, the adjustable arm lock 218 will be biased out of engagement with the first set of teeth 204 when the adjustable arm 212 is urged away from the fixed arm 208, but not when urged in the opposite direction. Thus, the adjustable arm 212 can be moved away from the fixed arm 208 by turning the adjustable arm thumbwheel 216 to open the hinged retractor 100, but will secure the hinged retractor 100 open in place during a procedure. To move the adjustable arm 212 toward the fixed arm 208, for example, to close the hinged retractor 100 for removal, the adjustable arm lock 218 must be separately urged open, and then the adjustable arm thumbwheel 216 turned in the desired direction. As one example of an alternative, a blade spreader could include two adjustable arms instead of one adjustable and one fixed arm.

The blade spreader 200 also includes a securement assembly 220. The securement assembly 220 is used to position and secure the securement bar 202 (and consequently the fixed arm 208 and adjustable arm 212, along with the hinged retractor 100 which is secured to them) relative to a frame that is fixed, for example, to the bed of the patient. The securement assembly 220 may include a securement clamp 220, a securement thumbwheel 224, and a securement lock 226. The securement clamp 220 is used to fix the securement assembly 220 to, for example, a frame such as the frame 30 that in turn is secured to a hospital bed. The securement thumbwheel 224 and securement lock 226 may be designed generally similar to the adjustment arm thumbwheel 216 and adjustable arm lock 218, and function in a generally similar manner to cooperate with the second set of teeth 206 to position the spreader bar 202. Thus, turning of the securement thumbwheel 224 in a given direction moves the fixed arm 210 (which is secured in place relative to the spreader bar 202) laterally relative to the securement clamp 220 (which is secured in place to the frame).

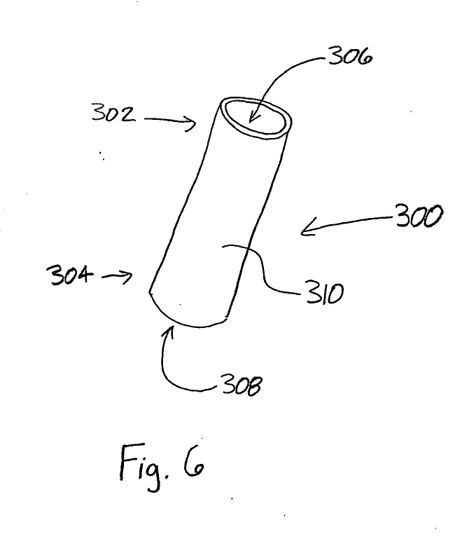

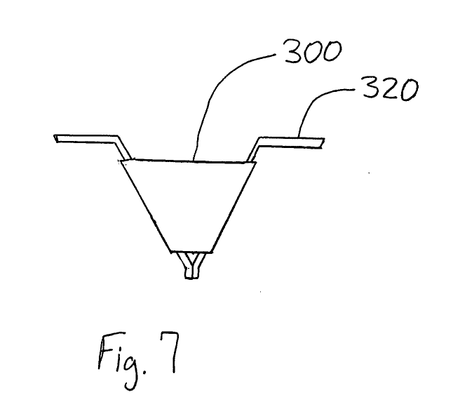

FIG. 6 illustrates a perspective view of a sheath 300 formed in accordance with an embodiment of the present invention, and FIG. 7 illustrates a side view of the sheath 300 in place on a hinged retractor in accordance with an embodiment of the present invention. The sheath 300 is used to help prevent or minimize the entry of tissue around the sides of the blades of a hinged retractor into a site of interest, thereby helping provide better access to the site. The sheath 300 illustrated in FIG. 6 includes a proximal end 302, a distal end 304, a first opening 306, a second opening 308, and a wall 310. Regarding the proximal end 302 and a distal end 304, “proximal” and “distal” are generally used in the same sense as described above. The first opening 306 is located proximate to the proximal end 302, and the second opening 308 is located proximate to the distal end 304. The sheath 300 may made out of an elastic, or stretchable material, with the dimension of the sheath 300 and the thickness of the wall 310 sized to fit snugly over a hinged retractor when the hinged retractor is in the closed position, and to expand with the hinged retractor when the hinged retractor is moved to the open position, while still providing sufficient resiliency to resist the incursion of tissue around the sides of the blades of the hinged retractor. Thus, the sheath 300 acts to provide a barrier around and between the blades of the hinged retractor to prevent tissue from entering the access site. The sheath 300 may be made, for example, from a latex free surgical grade elastic material. Further, the sheath and retractor blades may have cooperating surfaces and/or finishes to help maintain the position of the sheath after it is placed on the hinged retractor. FIG. 7 illustrates a sheath 300 snugly fitting around a hinged retractor 320 with the hinged retractor 320 in an open position.

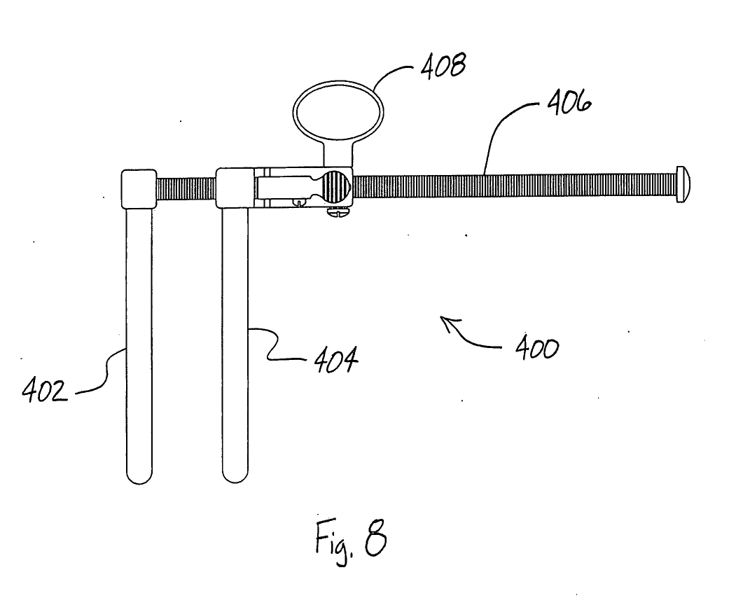

FIG. 8 illustrates a side view of a sheath spreader 400 formed in accordance with an embodiment of the present invention. The sheath spreader 400 is used to spread an opening of the sheath 300 to ease insertion of the hinged retractor 100 into an opening of the sheath 300. The sheath spreader 400 of the illustrated embodiment includes a first arm 402, a second arm 404, teeth 406, and a thumbwheel 408. The sheath spreader 400 is sized and adapted to stretch a sheath sufficiently to allow insertion of a hinged retractor. For example, the overall width of the sheath spreader may be approximately 8⅜ inches. Turning the thumbwheel 408 actuates the second arm 404 laterally relative to the first arm 402 (the first arm 402 is fixed relative to the teeth 406). As will be appreciated by one skilled in the art, alternate mechanisms may be used to actuate the second arm 404 laterally with respect to the first arm 402. To use the sheath spreader 400, the second arm 404 is positioned at or near the first arm 402. The free ends of the first arm 402 and the second arm 404 are then inserted into an opening of the sheath 300. Once the sheath 300 is in place on the sheath spreader 400, the thumbwheel 408 is turned to move the second arm 404 away from the first arm 402, until the sheath 300 is sufficient spread to accept the hinged retractor 100. The hinged retractor 100, in a closed position, is then inserted into an opening of the sheath 300, and advanced to a desired position. The thumbwheel 408 is then turned to bring the second arm 404 closer to the first arm 402. As the second arm moves 404 continues to moves toward the first arm 402, the sheath 300 collapses toward a snug fit around the blades of the hinged retractor 100, and the sheath spreader 400 may be removed.

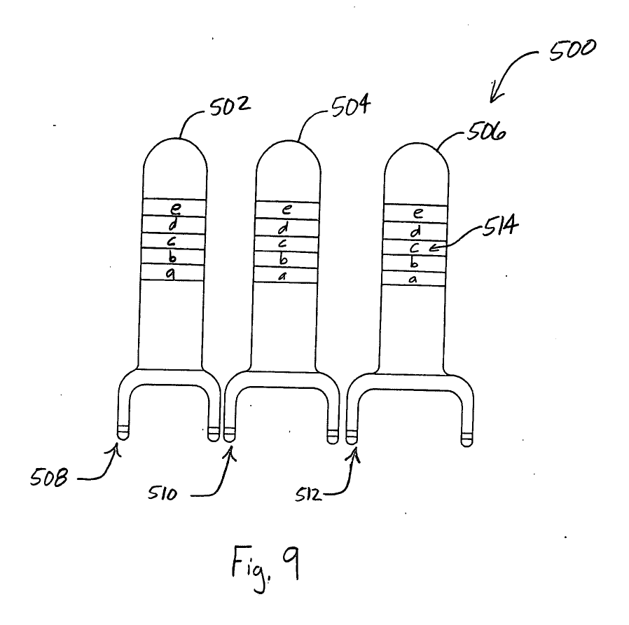

FIG. 9 illustrates a blade gauge set 500 formed in accordance with an embodiment of the present invention. A blade gauge may be used to determine an appropriate hinged retractor for use with a given patient and procedure, by helping determine an appropriate shape for the distal end of the hinged retractor, and/or determining the location at which the handle of the hinged retractor may be bent. As the anatomies of patients will vary across the population, different sizes and shapes of hinged retractors will work better for various patients and procedures. Use of such blade gauges may help save time and money in performing a given procedure for a given patient by making selection of an appropriate hinged retractor more efficient. For example, use of multiple hinged retractors by trial and error to select an appropriate shape may result in having to dispose of each hinged retractor that a surgeon attempts to place in a given patient, causing an increase in both the expense of the procedure and the time required to select an appropriate retractor. In the illustrated embodiment, the gauge set 500 includes a first gauge 502, a second gauge 504, and a third gauge 506. The first gauge 502 includes a first profile 508, the second gauge 504 includes a second profile 510, and the third gauge 506 includes a third profile 512. Each of the profiles are dimensioned and/or shaped differently from the other profiles, and each profile corresponds to a similarly shaped and dimensioned hinged retractor. For example, the first profile 508 may include a cutout width of approximately 50 millimeters, the second profile 510 may include a cutout width of approximately 57.5 millimeters, and the third profile 512 may include a cutout width of approximately 65 millimeters. A surgeon may determine an appropriate profile by comparing the profiles of the gauges to an x-ray of the site of interest and/or by inserting the gauge into the patient near the site of interest before placing the hinged retractor. Once an appropriate profile has been determined by use of the gauge set 500, a hinged retractor corresponding to that profile may be selected for the procedure.

In the embodiment illustrated in FIG. 9, each of the first, second, and third gauges 502, 504, 506 include depth indicators 514. The depth indicators 514 are used to determine an appropriate depth for bending a handle of a corresponding hinged retractor. For example, a gauge as illustrated in FIG. 9 may include depth indicators 514 corresponding to the depth indicators 134 of the blade 120 illustrated in FIGS. 3-4. After an appropriate gauge has been selected based on profile, that gauge may be placed in the patient, with a distal end of the gauge positioned at the site of interest similar to how the blade of a hinged retractor would be positioned. Then, based on a visual inspection of the protrusion of a handle of the gauge extending out of the patient, one of the depth indicators 514 of the gauge may be chosen to provide a bend at an elevation that will be convenient to mount to a mounting frame and/or a bar spreader as described above, as well as provide the desired clearance above the patient of the bent portion of the handle, and desired access to the site of interest. Once this determination has been made, the hinged retractor may be bent at the selected corresponding one of the depth indicators 134 of the hinged retractor. Thus, the hinged retractor may be pre-bent to shape before insertion into a patient, providing for increased convenience and ease of use of the hinged retractor.

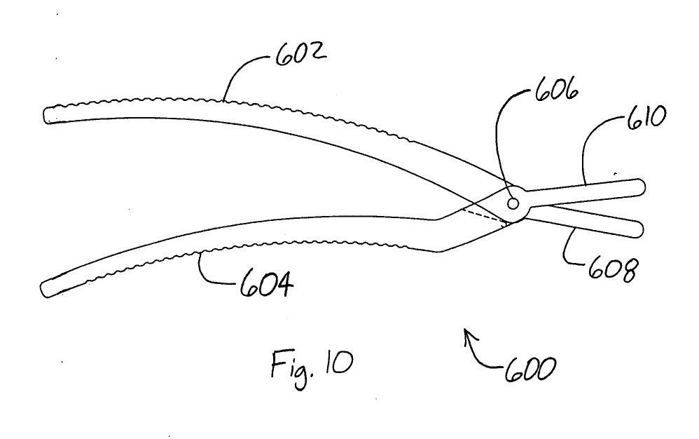

FIG. 10 illustrates a blade bender 600 formed in accordance with an embodiment of the present invention. The blade bender 600 includes a first arm 602 and a second arm 604 joined by a pivot 606. The first and second arms 602, 604, may be used to grasp a hinged retractor blade to be bent in a scissoring action. For example, the first arm 602 may include a first prong 608, and the second arm 604 may include two second prongs 610 disposed laterally on either side of the first prong 608. The prongs 608, 610 are sized and adapted to grasp a hinged retractor blade to be bent, and the first and second arms 602, 604 are sized and adapted to provide a comfortable fit in the hand of a practitioner as well as to proved leverage for bending a retractor blade. For example, the overall length of the blade bender 600 may be approximately 14 inches. To use the blade bender 600, a hinged retractor blade is grasped between the first prong 608 and second prongs 610 at or near a desired point of bending. Squeezing the first arm 602 and second arm 604 together secures the blade between the first prong 608 and the second prongs 610. Once grasped in the blade bender 600, the blade may be bent to a desired shape.

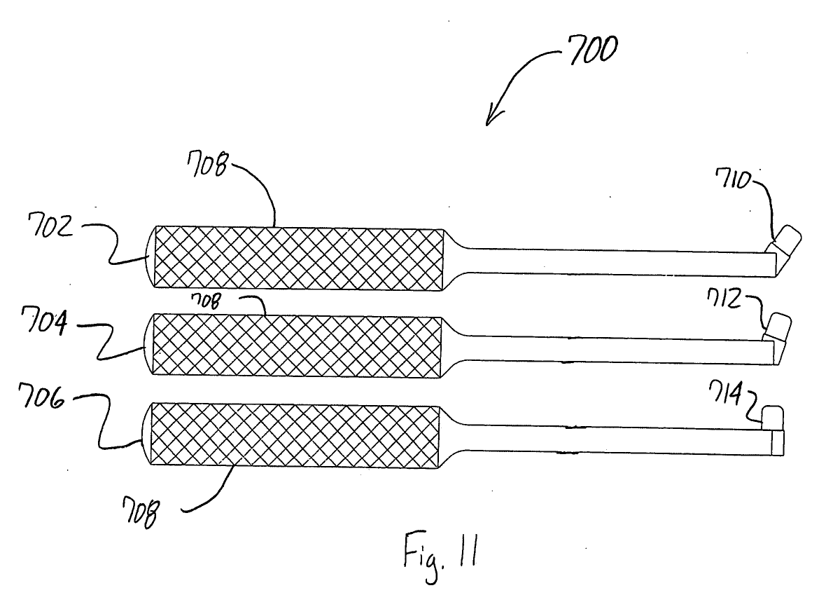

FIG. 11 illustrates a teeth bender set 700 formed in accordance with an embodiment of the present invention. The teeth bender set 700 may be used to help bend and position the teeth of a hinged retractor. For example, the teeth bender set 700 may be used to bend the teeth 128 of the blade 120 illustrated in FIGS. 3-4. In the embodiment illustrated in FIG. 11, the teeth bender set 700 includes a first tooth bender 702, a second tooth bender 704, and a third tooth bender 706. Each of the tooth benders 702, 704, 706 includes a handle 708 sized and adapted to be grasped by a surgeon and to provide leverage to help bend the teeth of a hinged retractor. For example, the overall length of each tooth bender may be approximately 6¾ inches. The first tooth bender 702 includes a first end 710, the second tooth bender 704 includes a second end 712, and the third tooth bender 706 includes a third end 714. In the illustrated embodiment, each of the ends is sized and adapted to be accepted by a bending tool opening 130 of the blade 120. The ends may be formed at different angles to facilitate bending of teeth to or from different positions, and/or provide differing angles of access to a bending tool opening 130. For example, the first end 710 may be formed at an angle of 125 degrees to the handle 708 of the first tooth bender 702, the second end 712 may be formed at an angle of 108 degrees to the handle 708 of the second tooth bender 704, and the third end may be formed at an angle of 90 degrees to the handle 708 of the third tooth bender 706.

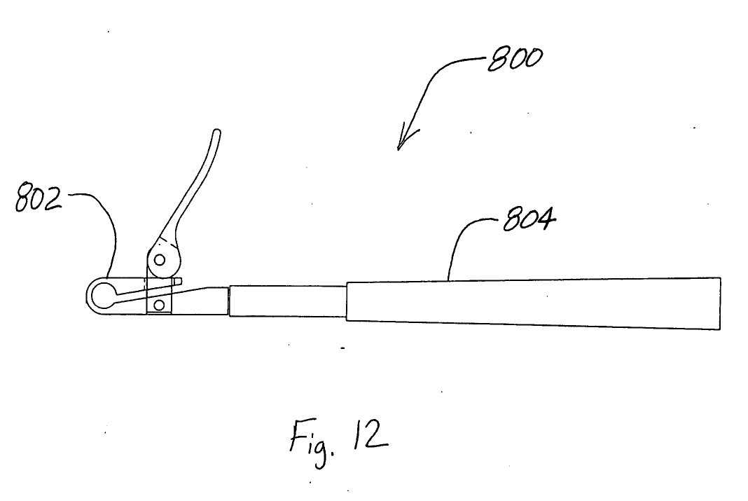

FIG. 12 illustrates a blade manipulator 800 formed in accordance with an embodiment of the present invention. The blade manipulator 800 may be used to ease insertion and/or positioning of a hinged retractor. The blade manipulator 800 illustrated in FIG. 12 includes a manipulator attachment member 802 and a handle 804. The manipulator attachment member 802 is adapted to securely grasp and release a portion of a hinged retractor. For example, the manipulator attachment member 802 of the embodiment illustrated in FIG. 12 includes a clamp sized to accept and secure the handling member 138 of the blade 120 illustrated in FIGS. 3-4. The handle 804 is shaped to provide a convenient grasping surface in a practitioner's hand, and sized to provide adequate leverage for positioning a hinged retractor. For example, the overall length of the blade manipulator 800 may be approximately 8 inches.

With reference to the above discussion, an example of the use of a hinged retractor formed in accordance with an embodiment of the present invention will now be described, in connection with the use of the hinged retractor 100 of the illustrated embodiment during a spinal fixation procedure to install screws in a patient's spine and/or to fix a rod connecting screws installed in the spine. An incision is first made in the patient. Then an appropriate gauge from the gauge set 500 is placed at the site of interest. The appropriate gauge is selected based on the profile of the gauge as compared to an x-ray of the site of interest and/or trial and error insertion of gauges at or near the site of interest, and an appropriate hinged retractor 100 corresponding to the profile of the selected gauge is chosen. With the gauge inserted in the patient, an appropriate bending point of the blades of the hinged retractor 100 is determined using the depth indicators 514. Once the appropriate bending point is determined, the blades of the hinged retractor 100 are bent, using the blade bender 600, to the desired shape at or near the depth indicator 134 corresponding to the depth indicator 514 determined by the use of the gauge. Thus, a gauge may be used to determine an appropriate hinged retractor by helping determine a desired profile and/or a desired bending location and shape.

Next, with the hinged retractor 100 in a closed position, a sheath 300 is attached to the hinged retractor 100. With the sheath 300 spread by the sheath spreader 400, the distal portion of the hinged retractor 100 is inserted into the sheath as described previously. After the sheath 300 is in place and with the hinged retractor 100 in the closed position, the blade manipulator 800 is attached to the hinged retractor 100, and the hinged retractor 100 is inserted into the desired position in the patient. The hinged retractor 100 may be inserted until, for example, the pivot 110 is located at or near a point beneath the lamina of the vertebrae being accessed, with the cutouts of the blades of the hinged retractor 100 positioned to provide an opening to the adjacent vertebrae being accessed.

Next, the blade spreader 200 is positioned and secured to the frame 30. The fixed arm 208 and the adjustable arm 212 are next positioned in place proximate to the securement member 140 of each blade of the hinged retractor 100 and secured thereto. The blade manipulator 800 is next removed from the hinged retractor 100, and the adjustable arm thumbwheel 216 turned to separate the fixed arm 208 and the adjustable arm 212. The arms of the blade spreader 200 are spread to the desired position and secured in place. This spreading of the arms opens the hinged retractor 100, holding back tissue and providing access to the site of interest. This allows for access to the site of interest, while also providing for a larger access area permitting greater ease of use of implements as well as improved lighting over so-called “minimally invasive” procedures. As the arms are spread and the hinged retractor 100 moves to an open position, the sheath 300 expands with the blades and helps prevent the incursion of tissue between the blades.

During and/or after the spreading of the arms of the blade bender 200, the position of the hinged retractor 100 may be biased away from its desired position. To correctly maintain the desired position, appropriate adjustments may be made by turning the securement thumbwheel 224 of the securement assembly 220 to correctly position the spreader bar 202. As one example of an alternative, in certain embodiments the hinged retractor 100 may be opened manually and mounted to a conventional retractor frame and clamps using methods known in the art.

With the hinged retractor 100 in the open position and secure, improved access to the site of interest may be obtained by bending the teeth 128 of the blade 120 to appropriate positions using the teeth bender set 700. The site may now be accessed to attach the required screw and/or rod for the fixation procedure.

While particular embodiments of the invention have been shown, it will be understood that the invention is not limited thereto since modifications may be made by those skilled in the art, particularly in light of the foregoing teaching. It is therefore, the appended claims that define the true spirit and scope of the invention.

What is claimed is:

1. A hinged retractor comprising a distal portion adapted to be located inside the body of a patient during a surgical procedure and a proximal portion adapted to be closer to the surgeon during a surgical procedure, said hinged retractor comprising
a first side including a first blade;
a second side including a second blade, said second side joined to said first side proximate to a distal end of said hinged retractor; and,
a pivot proximate to said distal end, wherein said hinged retractor is movable between a closed position and an open position, wherein a distance between a proximal portion of said first and second blades increases more than a distance between a distal portion of said first and second blades when said hinged retractor is moved from said closed position to said open position.

2. The hinged retractor of claim 1 comprising a sheath attachable to and removable from said first and second blades, said sheath when attached to said first and second blades surrounding at least a portion of said distal portion of said hinged retractor, said sheath being expandable with said hinged retractor when said hinged retractor is moved from said closed position to said open position and collapsible with said hinged retractor when said hinged retractor is moved from said open position to said closed position.

3. The hinged retractor of claim 1 wherein at least one of said first and second blades comprises a plurality of depth indicators, said plurality of depth indicators indicating positions at which said at least one of said first and second blades may be bent to adapt said hinged retractor for a procedure.

4. The hinged retractor of claim 1 wherein at least one of said first and second blades comprises a handling member for manipulating said hinged retractor during a procedure.

5. The hinged retractor of claim 1 wherein at least one of said first and second blades comprises bendable teeth proximate to said distal end of said hinged retractor.

6. The hinged retractor of claim 5 wherein at least one of said bendable teeth comprises an opening adapted for insertion of a bending tool for manipulating said at least one of said bendable teeth.

7. The hinged retractor of claim 1 wherein said first blade and second blade each include a respective first and second securement member adapted for mounting said first blade and second blade to a support structure for securing said first and second blade in position during a procedure.

8. The hinged retractor of claim 1 wherein said first and second blade are removably joined.

9. A retractor system comprising
a hinged retractor comprising a distal portion adapted to be located inside the body of a patient during a surgical procedure and a proximal portion adapted to be closer to the surgeon during a surgical procedure, said hinged retractor comprising
a first side including a first blade;
a second side including a second blade, said second side joined to said first side proximate to a distal end of said hinged retractor; and,
a pivot proximate to said distal end, wherein said hinged retractor is movable between a closed position and an open position, wherein a distance between a proximal portion of said first and second blades increases more than a distance between a distal portion of said first and second blades when said hinged retractor is moved from said closed position to said open position; and
a blade spreader securable to said first and second blades, said blade spreader adapted to maintain said hinged retractor in said open position during a procedure, said blade spreader securable to a frame for securing the retractor system in place during a procedure.

10. The retractor system of claim 9 wherein said blade spreader comprises a first adjustment portion adapted to open said hinged retractor and a second adjustment portion adapted to position said hinged retractor relative to said frame.

11. The retractor system of claim 9 further comprising a sheath attachable to and removable from said first and second blades, said sheath when attached to said first and second blades surrounding at least a portion of said distal portion of said hinged retractor, said sheath being expandable with said hinged retractor when said hinged retractor is moved from said closed position to said open position and collapsible with said hinged retractor when said hinged retractor is moved from said open position to said closed position.

12. The retractor system of claim 11 further comprising a sheath spreader adapted to expand said sheath to facilitate attachment of said sheath to said first and second blades.

13. The retractor system of claim 9 wherein at least one of said first and second blades comprises bendable teeth proximate to said distal end of said hinged retractor.

14. The retractor system of claim 13 further comprising a teeth bending tool, wherein at least one of said bendable teeth comprises an opening adapted for insertion of said teeth bending tool for bending said at least one of said bendable teeth.

15. The retractor system of claim 9 further comprising a gauge comprising a plurality of gauge depth indicators adapted to determine a bending location for at least one of said first and second blades, wherein the at least one of said first and second blades comprises a plurality of blade depth indicators corresponding to said gauge depth indicators, said plurality of blade depth indicators indicating positions at which said at least one of said first and second blades may be bent to adapt said hinged retractor for a procedure.

16. The retractor system of claim 9 further comprising a blade bender adapted to facilitate bending of at least one of said first and second blades.

17. A method of providing access to a surgical incision with a hinged retractor, the hinged retractor comprising a distal portion adapted to be located inside the body of the patient during a surgical procedure and a proximal portion adapted to be closer to the surgeon during a surgical procedure, wherein said hinged retractor is movable between a closed position and an open position, wherein a distance between a proximal portion of said first and second blades increases more than a distance between a distal portion of said first and second blades when said hinged retractor is moved from said closed position to said open position, the method comprising
positioning, inside the patient, the hinged retractor in said closed position;
adjusting the hinged retractor to said open position; and
securing the hinged retractor in place.

18. The method of claim 17 comprising surrounding at least a portion of the distal portion of the hinged retractor with an expandable sheath before said positioning of the hinged retractor inside the patient.

19. The method of claim 17 wherein the hinged retractor comprises at least one bendable tooth proximate to a distal end of the hinged retractor, comprising bending the at least one bendable tooth after said positioning of the hinged retractor inside the patient.

20. The method of claim 17 comprising using a gauge to determine an appropriate hinged retractor before said positioning of the hinged retractor inside the patient.
